(19) United States
(12) Patent Application Publication
(10) Pub. No.: US 2023/0372241 A1
(43) Pub. Date: Nov. 23, 2023

(54) SACCHARIDE-BASED ORAL MUCOADHESIVE DELIVERY SYSTEM FOR NUTRITIONAL AND NUTRACEUTICAL COMPOSITIONS

(71) Applicant: FP NUTRACEUTICALS, LLC, Santa Fe Springs, CA (US)

(72) Inventor: Timothy McCoy Marshall, Tucson, AZ (US)

(73) Assignee: FP NUTRACEUTICALS, LLC, Santa Fe Springs, CA (US)

(21) Appl. No.: 18/366,945

(22) Filed: Aug. 8, 2023

Related U.S. Application Data
(63) Continuation of application No. 16/673,038, filed on Nov. 4, 2019.
Application No. 16/673,038 is a continuation of application No. 16/128,349, filed on Sep. 11, 2018.
(60) Provisional application No. 62/696,955, filed on Jul. 12, 2018.

(51) Int. Cl. A61K 9/00; A61K 9/70; A61K 9/28; A61K 9/16

(52) U.S. Cl. CPC A61K 9/006; A61K 9/7007; A61K 9/2886; A61K 9/2826; A61K 9/1652

(57) ABSTRACT

The oral cavity is an ideal-site for small molecule, nutrient and/or nutraceutical delivery to the systemic circulation due to the highly-vascularized, oral mucosa, near-neutral pH conditions, and avoidance of gastric and first-pass degradation. Accordingly, novel, saccharide-based, food-form, portable, individually wrapped, oral delivery systems that attach to these four, highly absorptive surfaces (e.g. gingival, buccal, soft palatal, sublingual) for a longer contact-duration than current orally available formulas resulting in greater, small molecule bioavailability, and a more rapid, therapeutic effect are preferred and described.

### CROSS REFERENCE TO RELATED APPLICATIONS

This application claims priority from U.S. Provisional Patent Application No. 62/696,995, filed Jul. 12, 2018 and entitled “A NOVEL, SACCHARIDE-BASED, ORAL MUCOADHESIVE DELIVERY SYSTEM FOR FAST-ACTING, NEUROTROPHIC AND NEUROPROTECTIVE BENEFITS,” the disclosures of which are hereby incorporated entirely herein by reference.

### TECHNICAL FIELD

This disclosure generally relates to delivery systems, and more particularly to a saccharide-based oral mucoadhesive delivery system for administration of small molecule, neurotrophic, general or specialized nutritional supplements, and to nutrient and/or nutraceutical compositions.

### BACKGROUND

Mucoadhesion is a specific phenomenon of creating bonds during intimate contact between biological surfaces covered by a mucus layer and a mucoadhesive material. The oral bioavailability and uptake of small molecules (e.g. nutrients, nutraceuticals) is often limited by the short contact-time between the formulation and the oral mucosa, and a fast washout due to saliva flow.

Mucoadhesive dosage forms may be designed (e.g. soft chew) to enable prolonged retention for increased small molecule absorption for improved therapeutic outcomes, efficacy, and consumer benefit. Application of dosage forms to mucosal surfaces may be of benefit to small molecules not amenable to the oral route, such as those that undergo acid degradation or extensive first-pass metabolism. Mucoadhesive-based formulations have shown enhanced bioavailability. Mucoadhesive small molecule delivery provides rapid absorption and improved bioavailability due to considerable surface area and high blood flow. Small molecule delivery across the mucosa bypasses gastrointestinal acid and enzymatic degradation, and first-pass hepatic metabolism (Shaikh R, et al. J Pharm Bioallied Sci. 2011; 3(1):89-100).

Deficiencies of existing mucosal delivery systems include short mucosal contact time and prolonged time required for absorption of ionized or larger molecular-weight compounds.

Accordingly, what is needed is an improved mucosal delivery system, such as for neurotrophic and neuroprotective compositions, with prolonged mucosal contact time with more rapid mucosal absorption of the delivered composition than currently available systems.

### SUMMARY

In some embodiments, disclosed is a novel, highly-bioavailable, fast-acting, naturally-occurring, saccharide-based, food-form, oral (e.g. gingival, buccal, soft-palatal, sublingual), mucoadhesive, nutrient and/or nutraceutical delivery system for general nutritional support, for specific health concerns (e.g. cognitive impairment, sleep problems, tension), and a wide-range of CNS-related conditions and disorders. As previously described, this delivery system is designed to work synergistically with neurotrophic and neuroprotective, small molecule nutrients and nutraceuticals.

In some preferred embodiments, the composition is 100% naturally-derived (e.g. plant extracts) with a mucoadhesive, saccharide-base comprised of tapioca syrup, palm oil, non-GMO citric acid and/or malic acid and/or fumaric acid, sunflower lecithin, plant-extracted colors and flavors, and sea salt. In some embodiments, the naturally-derived, mucoadhesive, saccharide-base is comprised of tapioca syrup, palm oil and/or coconut oil, citric acid and/or malic acid and/or fumaric acid, sunflower or soy lecithin, natural and/or artificial flavors, and salt. In some embodiments, the natural (or synthetically-derived) saccharide-base is comprised of isomalto-oligosaccharide (IMO) syrup combined with palm oil and/or coconut oil, citric acid and/or malic acid and/or fumaric acid, soy and/or sunflower lecithin, natural and/or artificial flavors, natural and/or artificial colors, and salt.

In some embodiments, the mucoadhesive, saccharide-base is comprised of any one or combination of the following naturally-occurring (or chemically-identical, synthesized) saccharide complexes or isolates: tapioca syrup, isomalto-oligosaccharide (IMO) syrup, powdered isomalto-oligosaccharide (IMO), honey, powdered honey, yacon syrup, agave syrup, corn syrup, glucose syrup, coconut sugar syrup, coconut sugar, date syrup, molasses, rice syrup, sugar cane syrup, raw cane sugar, cane sugar syrup, turbinado syrup, allulose syrup, maltitol syrup, polyglycitol syrup, sugar beet syrup, inulin syrup, powdered inulin, fibrosol, maltodextrin, dextrin, gum arabic, dextrose anhydrous, dextrose monohydrate, dried glucose syrup, sorghum syrup, tagatose syrup, and the following sugar alcohols: erythritol syrup, mannitol syrup, sorbitol syrup, or xylitol syrup, ethylene glycol, glycerol, erythritol, threitol, arabitol, xylitol, ribitol, mannitol, sorbitol, galactitol, fucitol, iditol, and inositol combined with any combination of the following: palm oil, coconut oil, citric acid, malic acid, fumaric acid, tartaric acid, soy and/or sunflower lecithin, organic stevia (leaf) extract, monk fruit extract, artificial sweeteners (saccharin, acesulfame, aspartame, neotame, and sucralose), and natural and/or artificial flavors.

In some embodiments, the synergistic, CNS-directed, neurotrophic and/or neuroprotective preparation contains a magnesium chelate, or salt (e.g. magnesium oxide, magnesium sulfate, magnesium carbonate, magnesium chloride) as one of its components. In some embodiments, the magnesium chelating compound is chosen from the group consisting of succinic acid, ascorbic acid, aspartic acid, threonic acid, lysinic acid, malic acid, tauric acid, citric acid, and gluconic acid. In some embodiments, the magnesium chelating compound is a salt of orotic acid. In some embodiments, the magnesium chelate is a salt of glycine. For maximum therapeutic efficacy, in more preferred embodiments, the magnesium chelate should be one of the following: citrate, sulfate, or oxide (highly-ionized forms for treating constipation), glycinate, malate, taurate, threonate, or orotate.

In some embodiments, the synergistic, CNS-directed, neurotrophic and/or neuroprotective preparation comprises a zinc chelate, or salt (e.g. zinc oxide, zinc sulfate) as one of its components. In some embodiments, the zinc chelating compound is chosen from the group consisting of acetic acid, succinic acid, ascorbic acid, aspartic acid, threonic acid, lysinic acid, malic acid, tauric acid, citric acid, and gluconic acid. In some embodiments, the zinc chelating compound is a salt of orotic acid. In some embodiments, the zinc chelate is a salt of glycine. In some embodiments, the zinc chelate is a salt of methionine. For maximum therapeutic efficacy, in more preferred embodiments, the zinc chelate should be one of the following: citrate, glycinate, monomethionine, orotate, or acetate (highly-ionizable form of zinc for delivering Zn²⁺ ions directly to oral mucosal tissue for the effective treatment of viral throat infections).

In some embodiments, the synergistic, CNS-directed, neurotrophic and/or neuroprotective preparation comprises a lithium chelate or salt (e.g. lithium chloride, lithium citrate, or lithium carbonate) as one of its components. In some embodiments, the lithium chelating compound is chosen from the group consisting of succinic acid, ascorbic acid, aspartic acid, threonic acid, lysinic acid, malic acid, tauric acid, citric acid, and gluconic acid. In some embodiments, the lithium chelating compound is a salt of orotic acid. In some embodiments, the lithium chelating compound is a salt of aspartic acid. For maximum therapeutic efficacy, in more preferred embodiments, the lithium chelate should be one of the following: glycinate or orotate.

In some embodiments, the synergistic, CNS-directed, neurotrophic and/or neuroprotective preparation comprises an inorganic selenium salt (e.g. sodium selenate, sodium selenite), or more preferred, a selenium chelate or complex, as one of its components. In some embodiments, the selenium chelating compound is aspartate. In some embodiments, the selenium chelating agent is a non-specific amino acid or complex of amino acids (e.g. amino acid chelate). In some embodiments, the selenium chelating or complexing agent is the amino acid, methionine. In some embodiments, the selenium chelating or complexing agent is the amino acid, cysteine. In some, more preferred embodiments, the selenium chelating compound is glycinate. For maximum therapeutic efficacy, in more preferred embodiments, the selenium chelate or complexing agent should be one of the following: glycinate, methionine, cysteine, or high-selenium yeast.

These and other embodiments are further described in the detailed description and claims below. However, the claims are not intended to be limited to the embodiments and examples provided herein.

A more complete understanding of the present disclosure may be derived by referring to the detailed description and claims when considered in connection with the Figures, wherein like reference numbers refer to similar items throughout the Figures.

### BRIEF DESCRIPTION OF THE DRAWINGS

FIG. 1 is a diagram illustrating trans-mucosal absorption of a composition from the oral cavity;

FIG. 2 is a diagram illustrating a mucoadhesive delivery system used in a buccal and gingival position within the oral cavity;

FIG. 3 is a diagram illustrating a mucoadhesive delivery system used in a sublingual position within the oral cavity; and

FIG. 4 is a diagram illustrating a mucoadhesive delivery system used in a soft palatal position within the oral cavity.

### DETAILED DESCRIPTION OF EMBODIMENTS

The embodiments herein represent an oral mucoadhesive preparation with heretofore unrecognized benefits as a highly-efficacious, small molecule delivery system. In confections, a “fruit chew”, “chew”, of “soft chew” has been used for years in “Starburst-type” candies, and in the nutritional supplement industry, a form of it has been used, unwittingly without specific intention, other than as a palatable vehicle to carry a variety of nutritional substances (e.g. vitamins, minerals, nutraceuticals) without acknowledging, or realizing its true value and potential. To this date, there has been no discussion of its use, as an auspicious and effective mode of mucoadhesive, small molecule (e.g. nutraceutical, nutrient) delivery, which brings me to our present disclosure.

In one embodiment, a soft chew is provided with an extended mucoadhesive residence time, maximizing oral mucosal, small molecule absorption. Key components may include the addition of lecithin that boosts the mucoadhesive properties of the saccharide-base leading to a longer residence time, while palm oil that increases the elastic (spreadable) properties of the soft chew and increases the surface area of interaction, maximizing oral mucosal absorption. Mass range per chew is about 5.2-5.8 grams, with the average mass per chew being about 5.5 grams, wherein “about” means+/−10%. This allows for exceptional loading, with typical small molecule loading being in the range of 2.0-2.4 grams per chew.

In another embodiment, a “standard gummy” (containing predominately sugar, corn syrup, and/or glucose syrup, gelatin, and/or pectin) is provided with shorter mucoadhesive residence time that reduces oral mucosal small molecule absorption. Key components may include added pectin or gelatin as texturing agents that promote faster disintegration leading to a shorter residence time. This embodiment would lack the mucoadhesive enhancing properties of lecithin, and spreadable properties of palm oil, reducing oral mucosal absorption. Mass range per gummy is about 2.0-2.5 grams, with average mass per gummy being about 2.25 grams. This results in 2.4 times less carrying capacity than the average soft-chew, and typical small molecule loading is in the range of 0.8-0.9 grams per gummy.

In some embodiments, the amount of elemental magnesium per serving is between 10 and 400 milligrams per 1 soft-chew serving (˜5.2-5.8 grams/soft chew).

In some embodiments, the amount of elemental zinc per serving is between 2.5 and 50 milligrams per 1 soft-chew serving (˜5.2-5.8 grams/soft chew).

In some embodiments, the amount of elemental, nutritional lithium per serving is between 0.05 and 50 milligrams per 1 soft-chew serving (˜5.2-5.8 grams/soft chew).

In some embodiments, the amount of elemental selenium per serving is between 10 and 400 micrograms per 1 soft-chew serving (˜5.2-5.8 grams/soft chew).

In some embodiments, the amount of nutraceutical, phytocompounds is between 0.01 and 2400 milligrams per 1 soft-chew serving (˜5.2-5.8 grams/soft chew).

Disclosed is an oral mucoadhesive delivery system having a saccharide base; a natural color and/or artificial color or no color; and a natural flavor and/or artificial flavor or no added flavor.

In some embodiments, the saccharide base comprises tapioca syrup, palm oil, citric acid, and lecithin. In some embodiments, the saccharide base comprises isomalto-oligosaccharide (IMO) syrup, palm oil, citric acid, and lecithin. In some embodiments, the saccharide base comprises a saccharide from the group of saccharides consisting of: tapioca syrup, isomalto-oligosaccharide (IMO) syrup, powdered isomalto-oligosaccharide (IMO), honey, powdered honey, yacon syrup, agave syrup, corn syrup, glucose syrup, coconut sugar syrup, coconut sugar, date syrup, molasses, rice syrup, sugar cane syrup, raw cane sugar, cane sugar syrup, turbinado syrup, allulose syrup, maltitol syrup, polyglycitol syrup, sugar beet syrup, inulin syrup, powdered inulin, fibrosol, maltodextrin, dextrin, gum arabic, dextrose anhydrous, dextrose monohydrate, dried glucose syrup, sorghum syrup, tagatose syrup, and the following sugar alcohols: erythritol syrup, mannitol syrup, sorbitol syrup, or xylitol syrup, ethylene glycol, glycerol, erythritol, threitol, arabitol, xylitol, ribitol, mannitol, sorbitol, galactitol, fucitol, iditol, and inositol combined with any combination of the following: palm oil, coconut oil, citric acid, malic acid, fumaric acid, tartaric acid, soy and/or sunflower lecithin, organic stevia (leaf) extract, monk fruit extract, artificial sweeteners (saccharin, acesulfame, aspartame, neotame, and sucralose), natural and/or artificial flavors.

In some embodiments, the oral mucoadhesive delivery system further comprises a neurotrophic composition. In some embodiments, the neurotrophic composition comprises a chelate of a mineral from the group of minerals comprised of: magnesium, lithium, and zinc. In some embodiments, the neurotrophic composition comprises one or more nutraceuticals or phytocompounds (e.g. from hemp oil, echinacea, cacao) such as terpenes (e.g. limonene), phenolics (e.g. cyanidin), and various phytocannabinoids.

Disclosed is an oral mucoadhesive delivery system comprising a saccharide base; and a neurotrophic composition.

In some embodiments, the mucoadhesive delivery system also comprises a chelating compound. In some embodiments, the chelating compound is from the group of chelating compounds consisting of: succinic acid, ascorbic acid, aspartic acid, threonic acid, lysinic acid, orotic acid, malic acid, tauric acid, citric acid, and gluconic acid.

In some embodiments, the mucoadhesive delivery system comprises magnesium. In some embodiments, the mucoadhesive delivery system comprises zinc. In some embodiments, the mucoadhesive delivery system comprises lithium.

In some embodiments, the neurotrophic composition comprises selenium; and wherein the chelating compound is from the group of chelating compounds consisting of: aspartate, amino acid chelate, glycinate, methionine, and cysteine.

In some embodiments, the oral mucoadhesive delivery system further comprises a small molecule nutrient from the group of small molecule nutrients consisting of, in their most general forms: vitamin B1, Vitamin B2, vitamin B3, vitamin B5, vitamin B6, vitamin B9, vitamin B12, PABA, vitamin C, Vitamin D, Vitamin E, boron, calcium, chromium, copper, magnesium, manganese, molybdenum, potassium, selenium, sodium, strontium, vanadium, and zinc.

In some embodiments, the oral mucoadhesive delivery system further comprises a small molecule nutrient from the group of small molecule nutrients consisting of, in their most preferred (most bioavailable) forms: vitamin B1 (thiamine mononitrate and/or hydrochloride), Vitamin B2 (riboflavin and/or riboflavin-5-phosphate), vitamin B3 (niacinamide and/or nicotinic acid), vitamin B5 (pantothenic acid and/or calcium pantothenate), vitamin B6 (pyridoxine hydrochloride and/or pyridoxal-6-phosphate), vitamin B9 (folic acid, folinic acid, and/or methylfolate), vitamin B12 (adenosylcobalamin, hydroxocobalamin, and/or methylcobalamin), PABA, vitamin C (ascorbic acid, mineral ascorbates, ascorbyl palmitate), Vitamin D (cholecalciferol), Vitamin E (natural d-alpha tocopheryl succinate or acetate), boron glycinate, calcium ascorbate, chromium polynicotinate (dinicotinate-glycinate or picolinate), copper glycinate, magnesium malate (taurinate and/or glycinate), manganese glycinate, molybdenum glyicnate, potassium citrate, selenium glycinate (selenocysteine, selenomethionine, and/or high selenium yeast), sodium chloride, strontium chloride, vanadium (vanadyl sulfate), and zinc glycinate (citrate, monomethionine, and/or zinc amino acid chelate).

In some embodiments, the oral mucoadhesive delivery system is formed into a soft-chew or a gummy.

In some embodiments, the oral mucoadhesive delivery system is formed into an individually wrapped, soft-chew or a gummy.

Disclosed is an oral mucoadhesive delivery system comprising a saccharide base; a natural color; a natural flavor; and a neurotrophic composition having an elemental substance from the group of elemental substances consisting of magnesium, zinc, lithium, and selenium.

In some embodiments, the amount of elemental zinc per serving is between about 2.5 milligrams and about 50 milligrams. In some embodiments, the amount of elemental lithium per serving is between about 0.05 milligrams and about 50 milligrams. In some embodiments, the amount of elemental selenium per serving is between about 10 micrograms and about 400 micrograms.

As noted herein above, embodiments herein relate to a novel, fast-acting, highly-bioavailable, food-form (food-grade), saccharide-based (e.g. mono-, oligo-, polysaccharide), neurotrophic and neuroprotective, nutrient and/or nutraceutical delivery system 100 for a wide range of nutritional and medical uses whereby a quick-acting, portable, individually-wrapped, oral mucosal mode of delivery (i.e. soft chew) would be desirable and advantageous.

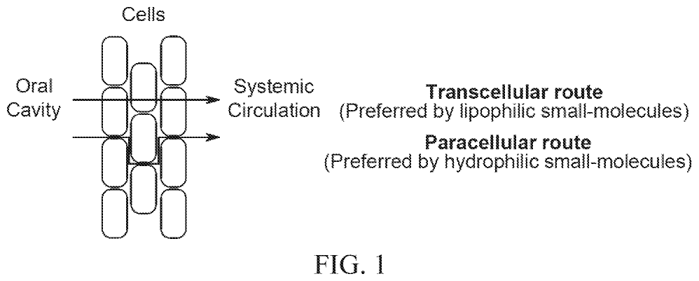

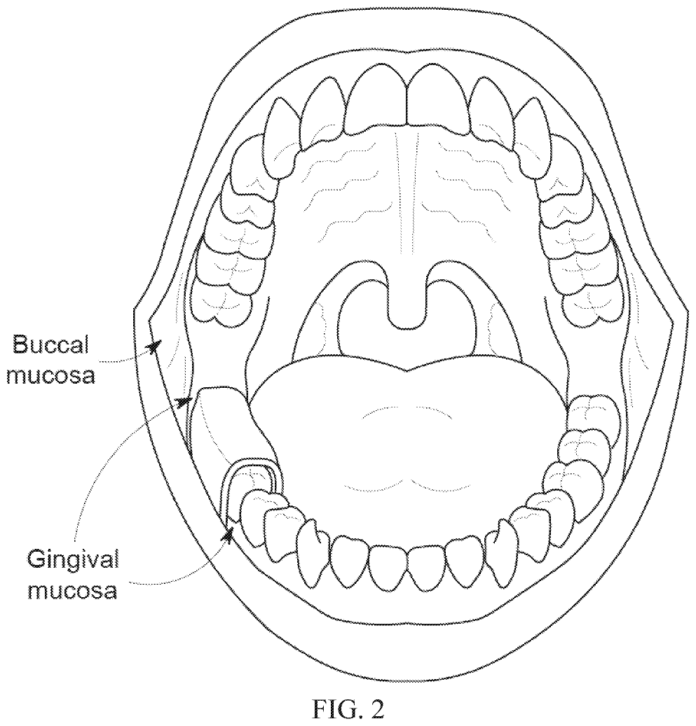

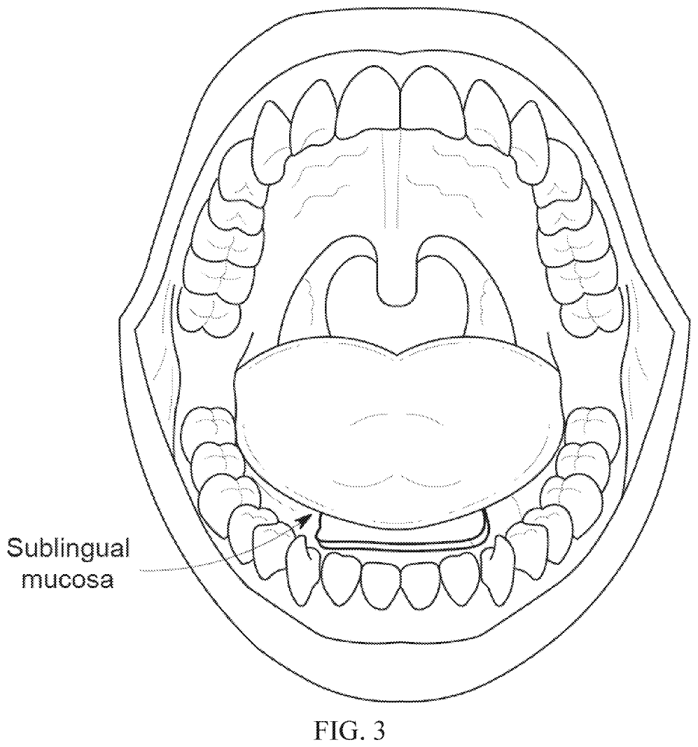

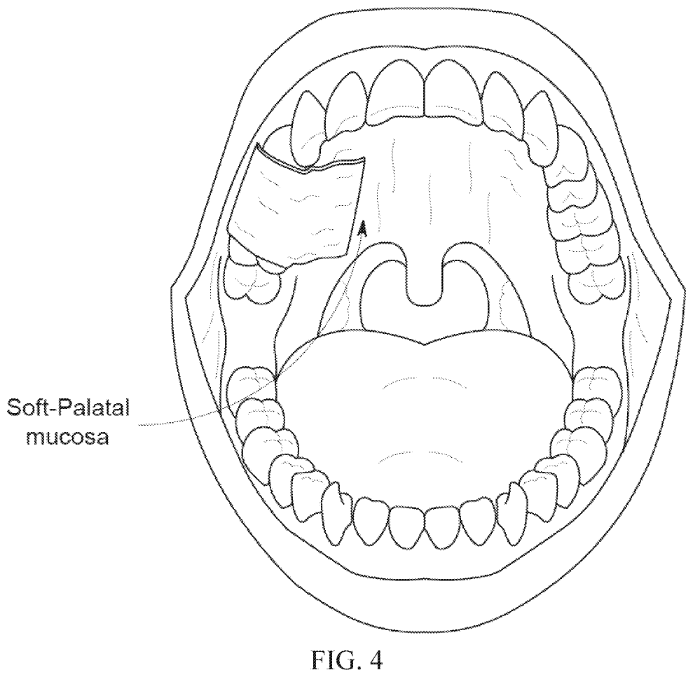

Through its innate, residence (contact-exposure) increasing, mucoadhesive properties—the novel, saccharide-based delivery system 100 efficiently increases small molecule, paracellular and transcellular transport (see FIG. 1) across four, highly-vascularized, oral-mucosal surfaces (e.g. gingival, buccal, sublingual, soft-palatal) for rapid systemic delivery and therapeutic onset (see FIG. 2-4). Due to its rich supply of blood vessels, near-neutral pH, and close proximity to the brain—the oral-mucosal tissues represent an ideal site for systemic, small molecule delivery—especially for CNS-related conditions involving depression, anxiety, sensitivity to stress, attention deficit hyperactivity disorder (“ADHD”), cognitive impairment, dementia, Alzheimer's disease, Parkinson's disease, neuroinflammation, NMDA-receptor hyperactivity, endocannabinoid deficiency, heavy-metal toxicity, chronic pain, insomnia, and sleep disturbances where rapid therapeutic intervention and onset is desired.

Given the biodynamics of molecular transport being largely a function of a molecule's size (molecular weight; mol wt), charge, and lipophilicity—it is a well-known pharmacological/nutrient principle that uncharged (neutral), smaller molecules traverse both the paracellular and transcellular transport routes into the systemic circulation more readily than charged, larger molecules. This dramatic effect of molecular size/weight on bioavailability can be readily seen in the small molecule, vitamin B6 (pyridoxine) which has a molecular weight of 169 Da and a bioavailability of 75-100% from food or supplements.

In contrast, and in alignment with the effect of molecular weight on bioavailability, the large-molecule, vitamin B12 (cobalamin) with a molecular weight of 1355 Da possesses an extremely low bioavailability of 1-2%. These two examples provide a general, quantitative template that can be applied to a wide variety of organic molecules of similar size/weight that clearly demonstrates the strong inverse relationship of a molecule's size/weight on mucosal absorption (i.e. the ability of a small molecule to traverse the oral and GI mucosa by simple, passive diffusion) and bioavailability.

Small molecule bioavailability and therapeutic onset are two important factors in the effectiveness of any nutritional or nutraceutical with intended, CNS-directed, neurotrophic or neuroprotective therapeutic benefits (e.g. antidepressant effect, anxiolytic, stress-reducing effect, calming effect, anti-inflammatory effect, antioxidant activity, reduction of oxidative-stress, pain relief, NMDA receptor inhibition), or for any indication where immediate therapeutic relief and benefit is desired.

For CNS-related conditions such as depression, anxiety, brain and nerve injury, headaches, chronic pain, and cognitive impairment where nutrient deficiencies and neuro-inflammation play a strong, contributing role—nutrient bioavailability and onset of therapeutic effect are of great value to the consumer. Non-toxic, technologies that improve bioavailability and speed of therapeutic onset are thus highly desirable characteristics in any nutritional, nutraceutical, or pharmaceutical product that has the ability to produce a valuable therapeutic effect.

A “sticky,” mucoadhesive, food-form, saccharide-based, “soft-chew” or “gummy” delivery system has three primary advantages over current oral technologies—(e.g. quick-dissolve tablets, non-mucoadhesive sublingual tablets, non-mucoadhesive lozenges, powder packets for liquid delivery)—designed for greater nutrient/nutraceutical bioavailability.

Advantages of the novel delivery system 100 include: 1. Increased residence time (i.e. extended contact-duration, exposure time of nutrients, directly in contact with the oral mucosa): due to the “sticky”, extended-oral-mucosal-contact-duration and multi-surface interaction (e.g. buccal, gingival, sublingual, soft-palatal) coupled to the close proximity to the brain—this novel, delivery system provides rapid, therapeutic onset for CNS-related conditions involving depression, anxiety, sensitivity to stress, attention deficit hyperactivity disorder (“ADHD”), cognitive impairment, neuroinflammation, NMDA-receptor hyperactivity, endocannabinoid deficiency, heavy-metal toxicity, chronic pain, and sleep disturbances where rapid therapeutic intervention and onset is desired.

This next-generation, oral delivery system exploits the highly-vascularized, oral mucosa, near-neutral pH, and close proximity to the brain to quickly and efficiently transport stress-reducing (i.e. anxiolytic), mood-boosting (i.e. anti-depressant), anti-inflammatory, small molecule nutrients (e.g. selenium glycinate, zinc glycinate, lithium orotate), nutraceuticals (e.g. phytocompounds: terpenes, phenolics, cannabinoids), and other similarly therapeutic, small molecule nutritional elements for maximum consumer benefit and therapeutic effect.

2. Unique biodynamics of delivery system: due to the bulky (˜5.2-5.8 g) and “sticky” nature of each “soft-chew” or “gummy”—when chewed, the mucoadhesive “soft-chew” or “gummy” spreads and extends over the upper and lower teeth, adhering to—making direct and prolonged contact with the gums (gingiva), inner sides of the cheeks (buccal), underside of the tongue (sublingual), and roof of the mouth (soft-palatal) (FIGS. 2-4). Average mass per chew: 5.5 grams. Allows for exceptional loading; typical small molecule loading is in the range of 2.0-2.4 grams per chew. Greater small molecule loading increases the concentration gradient at the mucoadhesive/mucosal interface for greater passive absorption.

3. Synergistic mucoadhesive, small molecule delivery system: this novel, mucoadhesive delivery system works synergistically with the following therapeutic, small molecule nutrients (mol wt<500 Da): lipophilic, neutral (uncharged), stable (poorly-ionized), mineral chelates (e.g. glycinates, mol wt<250 Da; lithium orotate, mol wt=162 Da, respectively), retinol (mol wt: 286 Da), thiamine (mol wt: 265 Da), riboflavin (mol wt: 376 Da), niacinamide (mol wt: 122 Da), pantothenic acid (mol wt: 219 Da), pyridoxine (mol wt: 169 Da), folinic acid (mol wt: 473 Da), folic acid (mol wt: 441 Da), vitamin C (mol wt: 176 Da), vitamin D (mol wt: 387 Da), and lipophilic, nutraceuticals such as terpenes (mol wt<230 Da), phenolics (mol wt<200 Da), and cannabinoids (mol wt<350 Da)—which possess greater bioavailability at the mucoadhesive-mucosa interface than larger, less-lipophilic molecules (e.g. zinc citrate, mol wt=574 Da).

For more rapid onset, and maximum therapeutic benefit and efficacy—lipophilic, uncharged, poorly-ionized, mineral chelates such as those bound to orotate or glycinate are the preferred vehicles (carriers) for efficient, mineral delivery and absorption (i.e. high-bioavailability). Small molecule, mineral chelates such as those bound to orotate (e.g. Li-orotate, mol wt: 162 Da; divalent ions, di-orotates of Ca, Mg, Zn<410 Da) or glycinate (e.g. bisglycinates of Ca, Mg, Cu, Mn, Mo, Se<250 Da) are absorbed via hydrophilic, paracellular transport and lipophilic, transcellular transport, and/or through transcellular, carrier-mediated orotate or glycine transporters.

A 1978 study comparing the bioavailability of lithium orotate to lithium carbonate demonstrated 3 times greater bioavailability for poorly ionized (lipophilic) lithium orotate as compared to highly ionized (hydrophilic) lithium carbonate. This study illustrates a primary distinction between the orotate vs carbonate forms of lithium, showing that the poorly-ionized, uncharged, orotate form more readily crosses lipophilic barriers (cell membranes) compared to the highly-ionized, charged, carbonate form—as evidenced by a 3-times higher level in the brain. With our present understanding of small-molecule bioavailability, this result can be reliably predicted and applied to a wide range of molecules with similar (low mol wt, neutral, uncharged, lipophilic; high-bioavailability) and dissimilar (high mol wt, ionized, charged, hydrophilic; low-bioavailability) physicochemical properties.

In further support of the effect of ionizability (i.e. ability to form charged ions) and molecular weight/size on bioavailability—a 2008 study investigating the acute uptake (ie. bioavailability) of four different forms of zinc (e.g. oxide, picolinate, gluconate, glycinate) found that zinc glycinate had the highest bioavailability. The following is the ionizability and molecular weight for each form: Zn-oxide (highly-ionized; mol wt: 81 Da), Zn-picolinate (moderately ionized; mol wt: 310 Da), Zn-gluconate (highly-ionized; mol wt: 456 Da), and Zn-glycinate (poorly-ionized; mol wt: 214 Da). Plasma zinc rankings based on area under the curve, as well as by rank results per person, were: glycinate>gluconate>picolinate=oxide. A +43.4% increase in bioavailability was seen for zinc glycinate over the second, most-bioavailable form, zinc gluconate.

In summary, lithium orotate (162 Da) and zinc glycinate (214 Da) demonstrate superior bioavailability due to their small size (low molecular weight) and both being stable, neutral chelates as compared to the unstable, highly ionized, charged variants (e.g. lithium carbonate, zinc oxide) of both forms.

Neutral, small molecule, nutrient/nutraceutical transport occurs through both a paracellular transport mechanism across the tight junctions between cells, and directly across the lipid-bilayer of cell membranes via transcellular transport. Their low-molecular weight (i.e. small size) favors and promotes paracellular transport, while their neutral charge (coupled with small size) permits highly efficient, transcellular (transmucosal) transport into the systemic circulation.

Stable, lipophilic, mineral chelates (e.g. glycinates, orotates) of magnesium, zinc, and lithium share similar physicochemical properties, in terms of size and lipophilicity, as that of known, highly-bioavailable, small molecules (mol wt<500 Da) such as caffeine (mol wt: 194 Da) and nicotine (mol wt: 162 Da)—with molecular weights between 162 Da (lithium orotate) to 214 Da (zinc glycinate). Nutraceuticals such as terpenes (e.g. limonene, 136 Da), phenolics (e.g. cyanidin, 287 Da), and cannabinoids (e.g. anandamide, 348 Da) also share similar physicochemical properties.

As discussed above, the disclosed embodiments relate to a novel, highly bioavailable, fast-acting, saccharide-based, multi-surface, oral (gingival, buccal, soft-palatal, sublingual), mucoadhesive delivery system for CNS-related conditions and disorders.

An example of a synergistic, neurotrophic and/or neuroprotective, small molecule, nutrient/nutraceutical preparation functioning in-tandem with the novel, mucoadhesive delivery system described herein might consist of (but is not limited to): magnesium glycinate, lithium orotate, zinc glycinate, thiamine, riboflavin, niacin, pantothenic acid, pyridoxine, folinic acid, cannabinoids (e.g. from hemp oil extract, echinacea, cacao), and various phytocompounds from plants (e.g. anthocyanins, terpenes, phenolics). Such a preparation would exert a number of neurotrophic and/or neuroprotective therapeutic benefits (e.g. antidepressant effect, stress-reducing effect, calming effect, anti-inflammatory effect, antioxidant activity, reduction of oxidative-stress, pain relief, NMDA receptor inhibition) in humans and animals.

The neurotrophic and/or neuroprotective preparation briefly described above functioning in biological synergy with the novel delivery system described herein—with its rapid therapeutic onset and effect—would be of great value and benefit to those with CNS-related conditions involving depression, anxiety, sensitivity to stress, attention deficit hyperactivity disorder (“ADHD”), cognitive impairment, dementia, Alzheimer's disease, Parkinson's disease, neuroinflammation, NMDA-receptor hyperactivity, endocannabinoid deficiency, heavy-metal toxicity, chronic pain, insomnia, and sleep disturbances where rapid therapeutic intervention is desired. The mucoadhesive, saccharide complex or isolate can be either naturally derived (e.g. tapioca syrup, rice syrup, isomalto-oligosaccharide (IMO) syrup) and/or chemically identical, synthetically derived.

The mucoadhesive properties of the saccharide complex or isolate is further enhanced with the addition of lecithin and palm oil. As a result of its innate mucoadhesive properties, lecithin boosts the mucoadhesive activity of the saccharide base leading to a longer residence time, while palm oil increases the elastic (spreadable) properties of the soft chew (increasing the contact surface; effective surface area of interaction)—together maximizing oral mucosal absorption.

Magnesium, lithium, zinc, and cannabinoids (e.g anandamide) exert a portion of their neuroprotective, antioxidant, anti-inflammatory effects through inhibition of the N-methyl-D-aspartate receptor (“NMDA”), with which magnesium (and/or lithium, zinc) interact as a receptor ligand, on post-synaptic cortical neurons of the central nervous system. With their innate antioxidant activity, and CB1/CB2 receptor binding affinity, cannabinoids are believed to exert their NMDA inhibitory effect through a different mechanism. NMDA receptors are ubiquitous throughout the brain and play a role in regulation of the excitatory state of post-synaptic neurons. NMDA receptors act as a cationic membrane “pore,” primarily for calcium ions although other cations such as sodium, zinc, and protons may pass into the cell.

In conditions wherein the post-synaptic neuron is polarized and glutamate is absent from the synapse, a local negative membrane charge permits the pore to be blocked with a magnesium ion. Under conditions wherein 1) glutamate is present within the synapse at a sufficient concentration; and 2) the post-synaptic neuron is partially depolarized creating a neutral or relative positive membrane charge, the magnesium ion is displaced, the pore opens, and calcium ions are allowed to pass freely through the NMDA receptor into the cell. Once intracellular, calcium exerts a myriad of secondary effects, largely through its role as a secondary messenger and enzyme cofactor. Increased intracellular calcium leads to increased cellular enzyme activity of proteases, nucleases, and phospholipases, breaking down structural components and functional machinery of the cell and often leading to cell death.

Keeping the baseline state of the NMDA receptor pore in a closed configuration, therefore, is important for the proper function and survival of a post-synaptic neuron. There are at least two potential sites of action to keep the receptor pore closed to influx of calcium and other cations into the neuron: 1) adequate-to-high synaptic magnesium concentrations; and 2) tyrosine-mediated phosphorylation of the NR2B receptor subunit.

Given a polarized or neutral post-synaptic cell membrane, in combination with adequate extracellular magnesium concentrations, magnesium binds to the receptor pore and blocks influx of calcium.

Several physiologic mechanisms resist a partially depolarized state in the post-synaptic cell membrane, keeping the pore closed to the influx of calcium an enhancing appropriate NMDA receptor function. One of these potentiating mechanisms is tyrosine-mediated phosphorylation of the NMDA receptor subunits, tending to “close” the receptor pore by causing an amphoteric shift in one or more protein subunits. Tyrosine phosphatase-mediated NR2B subunit phosphorylation potentiated by the lithium cation has been shown to cause depression of NMDA receptor currents.

The present embodiments, in addition to exceptional bioavailability with a broad-spectrum of neurotrophic effects (e.g. increased neural growth factors (e.g. BDNF), stem cell proliferation, Nrf2 activation, enhanced antioxidant defenses (e.g. GPx), essential nutrient repletion), seek to militate against activation of a final common pathway for neuronal cell injury and death-elevated intracellular calcium levels—by reducing oxidative stress and neural inflammation, and impeding permeability of the NMDA receptor to calcium.

Magnesium concentrations in a neuronal synapse are necessary to saturate the post-synaptic population of NMDA receptors, therein keeping the receptor pores closed to the influx of extracellular calcium ions when the post-synaptic neuron is in a partially polarized state. Hypomagnesaemia is associated with a plethora of symptomatic neurological abnormalities, such as depression, anxiety, sleep disturbances, hyperreflexia, tremor, confusion, hallucinations, convulsions, hyperacusis, nystagmus, tetany, delirium tremens, and extrapyramidal disorders.

Trans-mucosal absorption of elemental magnesium is greatly increased by combining elemental magnesium with a stable, organic chelate, which possesses greater lipophilicity, and thus greater mucosal uptake and bioavailability. In some embodiments, the magnesium chelating compound is a salt of glycine. In some embodiments, the magnesium chelating compound is a salt of orotic acid. In some embodiments, the magnesium chelating compound is a salt of succinic acid, aspartic acid, ascorbic acid, threonic acid, gluconic acid, lysinic acid, malic acid, tauric acid, or citric acid. It is anticipated that as experimentation and research in the art of enhanced trans-mucosal magnesium absorption progresses, other chelating compounds may be used as a chelating compound, in some embodiments.

The total concentration of elemental magnesium per dose of the neuroprotective preparation is calculated to provide adequate intracerebral levels of magnesium without being so high as to increase the risk of toxicity from hypermagnesaemia. Accordingly, the amount of elemental magnesium, in some embodiments, is between approximately 10 milligrams and 400 milligrams.

The synergistic nutrients and/or nutraceuticals used with this novel, mucoadhesive delivery system can by comprised, but not limited to, any one or combination of the following: vitamins, minerals, nutrients, or nutraceuticals used for their therapeutic, neurotrophic and/or neuroprotective benefits, as previously described above. In some embodiments, the proprietary neurosupportive blend comprises (per 1 soft-chew serving): magnesium as glycinate, malate, or taurinate (as well as other organic or inorganic forms of the mineral), 10-400 mg; kelp, 5-100 mg; vitamin A as retinyl palmitate or acetate or beta-carotene, 500-25,000 iu; vitamin B1, 0.5-100 mg; vitamin B2, 0.5-100 mg; vitamin B6, 0.5-100 mg; Vitamin B3, 0.5-250 mg; vitamin B5, 5-200 mg; Vitamin B12, 5-5,000 mcg as cyanocobalamin or methylcobalamin or hydroxocobalamin or adenosylcobalamin; folic acid or methyl-folate or folinic acid, 10-1,000 mcg; 25-1,000 mcg; non-GMO vitamin C, 25-1,000 mg; vitamin D3, 500-10,000 IU; vitamin E as d-alpha tocopherol or dl-alpha tocopheryl succinate or acetate and/or mixed tocopherols and/or tocotrienols, 5-1,000 iu for alpha-tocopherol and 5-500 mg for mixed tocopherols including gamma-tocopherol or the family of tocotrienols; boron as glycinate (as well as other organic or inorganic forms of the mineral), 0.5-10 mg; copper as bisglycinate (as well as other organic or inorganic forms of the mineral), 0.1-5 mg; zinc as bisglycinate (as well as other organic or inorganic forms of the mineral), 2.5-50 mg; manganese as bisglycinate (as well as other organic or inorganic forms of the mineral), 0.5-10 mg; selenium as glycinate (as well as other organic or inorganic forms of the mineral), 10-400 mcg; molybdenum as glycinate (as well as other organic or inorganic forms of the mineral), 10-1,000 mcg; and chromium as nicotinate glycinate (as well as other organic or inorganic forms of the mineral), 10-1,000 mcg; and nutraceuticals such as terpenes (e.g. limonene), phenolics (e.g. cyanidin), and cannabinoids.

The nutrients and/or nutraceuticals used in some embodiments are comprised of some of the most bioavailable nutrients (e.g. Albion® minerals) along with the novel, mucoadhesive delivery system previously described facilitating exceptional, oral bioavailability through the gingival, buccal, soft-palatal, and sublingual mucosa into the systemic circulation.

The pharmaceuticals used in some embodiments can be from a wide variety of categories and classes, and for various indications including but not limited to acute and chronic pain, fever, blood pressure regulation, and blood sugar management to name a few.

A novel, synergistic, fast-acting, highly bioavailable, saccharide-based, mucoadhesive, neurotrophic and/or neuroprotective delivery system for CNS-related conditions or disorders, and methods of use has been described. This disclosure represents the next-generation in optimized, highly efficacious, small molecule delivery systems for a wide variety of health issues, medical conditions, and CNS-related conditions that moves one-step closer in approximating intravenous injection in systemic bioavailability and therapeutic onset.

The description as set forth is not intended to be exhaustive or to limit the invention to the precise form disclosed. Many modifications and variations are possible in light of the teachings above.

1. An oral mucoadhesive delivery system comprising:
a plant-derived saccharide base; and
a sweetener.

2. The oral mucoadhesive delivery system of claim 1, wherein the saccharide base further includes lecithin.

3. The oral mucoadhesive delivery system of claim 1, wherein the saccharide-base further includes lecithin and palm oil.

4. The oral mucoadhesive delivery system of claim 3, further comprising a nutritional, multivitamin, or multi-nutrient composition selected from the group consisting of one of more of: vitamins A, B1, B2, B3, B5, B6, B9, B12, C, D2, D3, E, K1, K2, K3, essential fatty acids, amino acids, bioflavonoids, alpha lipoic acid, coenzyme Q10, boron, calcium, chromium, copper, iron, lithium, manganese, magnesium, molybdenum, nickel, selenium, sodium, strontium, and zinc.

5. The oral mucoadhesive delivery system of claim 4, further comprising a cannabinoid, terpene, or phenolic phytochemical.

6. The oral mucoadhesive delivery system of claim 4 formed into a soft-chew or a gummy.

7. The oral mucoadhesive delivery system of claim 4, wherein the zinc is elemental zinc and the amount of elemental zinc per serving is between about 2.5 milligrams and about 50 milligrams.

8. The oral mucoadhesive delivery system of claim 4, wherein the lithium is elemental lithium and the amount of elemental lithium per serving is between about 0.05 milligrams and about 50 milligrams.

9. The oral mucoadhesive delivery system of claim 4, wherein the selenium is elemental selenium and the amount of elemental selenium per serving is between about 5 micrograms and about 400 micrograms.

10. The oral mucoadhesive delivery system of claim 4, wherein the magnesium is elemental magnesium and the amount of elemental magnesium per serving is between 10 and about 400 milligrams.

11. The oral mucoadhesive delivery system of claim 5, the amount of phytochemcial is between 0.01 and 2400 milligrams per serving.

12. The oral mucoadhesive delivery system of claim 1, wherein the saccharide base comprises tapioca syrup, palm oil, citric acid, and lecithin.

13. The oral mucoadhesive delivery system of claim 12, wherein the saccharide base comprises a tapioca syrup, isomalto-oligosaccharide (IMO) syrup, powdered isomalto-oligosaccharide (IMO), honey, powdered honey, yacon syrup, agave syrup, corn syrup, glucose syrup, coconut sugar syrup, coconut sugar, date syrup, molasses, rice syrup, sugar cane syrup, raw cane sugar, cane sugar syrup, turbinado syrup, allulose syrup, maltitol syrup, polyglycitol syrup, sugar beet syrup, inulin syrup, powdered inulin, fibrosol, maltodextrin, dextrin, gum arabic, dextrose anhydrous, dextrose monohydrate, dried glucose syrup, sorghum syrup, tagatose syrup, and the following sugar alcohols: erythritol syrup, mannitol syrup, sorbitol syrup, or xylitol syrup, ethylene glycol, glycerol, erythritol, threitol, arabitol, xylitol, ribitol, mannitol, sorbitol, galactitol, fucitol, iditol, and inositol combined with any combination of the following: palm oil, coconut oil, citric acid, malic acid, fumaric acid, tartaric acid, soy and/or sunflower lecithin, organic stevia (leaf) extract, monk fruit extract, saccharin, acesulfame, aspartame, neotame, or sucralose.

14. The oral mucoadhesive delivery system of claim 1, wherein the oral mucoadhesive delivery system further comprises a neurotrophic composition selected from the group consisting of a limonene, a cyanidin, and a phytocannabinoid.

15. The oral mucoadhesive delivery system of claim 1, further comprising a chelating compound selected from the group of chelating compounds consisting of: succinic acid, ascorbic acid, aspartic acid, threonic acid, lysinic acid, orotic acid, malic acid, tauric acid, citric acid, and gluconic acid.

16. The oral mucoadhesive delivery system of claim 1, further comprising per serving: magnesium as glycinate, malate, or taurinate 10-400 mg; kelp, 5-100 mg; vitamin A as retinyl palmitate or acetate or beta-carotene, 250-25,000 iu; vitamin B1, 0.5-100 mg; vitamin B2, 0.5-100 mg; vitamin B6, 0.5-100 mg; Vitamin B3, 0.5-500 mg; vitamin B5, 5-500 mg; Vitamin B12, 5-10,000 mcg as cyanocobalamin or methylcobalamin or hydroxocobalamin or adenosylcobalamin; folic acid or methyl-folate or folinic acid, 10-10,000 mcg; non-GMO vitamin C, 10-2,000 mg; vitamin D3, 100-40,000 IU; vitamin E as d-alpha tocopherol or dl-alpha tocopheryl and/or mixed tocopherols and/or tocotrienols, 5-1,000 iu for alpha-tocopherol and 5-500 mg for mixed tocopherols including gamma-tocopherol or the family of tocotrienols; boron as glycinate 0.5-10 mg; copper as bisglycinate, 0.1-5 mg; zinc as bisglycinate, 2.5-50 mg; manganese as bisglycinate, 0.5-10 mg; selenium as glycinate, 5-400 mcg; molybdenum as glycinate, 10-1,000 mcg; and chromium as nicotinate glycinate, 10-1,000 mcg; and a terpene, phenolic, and cannabinoid.
